(19) United States
(12) Patent Application Publication
(10) Pub. No.: US 2024/0325367 A1
(43) Pub. Date: Oct. 3, 2024

(54) ROPIVACAINE SUSPENSION INJECTION, AND PREPARATION METHOD THEREFOR

(71) Applicant: ZHEJIANG CUIZE PHARMACEUTICAL TECHNOLOGY CO., LTD., Hangzhou (CN)

(72) Inventors: Siping SUN, Hangzhou (CN); Liang ZENG, Hangzhou (CN); Qiang GE, Hangzhou (CN); Dingqiang JIANG, Hangzhou (CN)

(21) Appl. No.: 18/554,118

(22) PCT Filed: Mar. 9, 2022
(86) PCT No.: PCT/CN2022/080019

§ 371 Date: Oct. 5, 2023

(30) Foreign Application Priority Data
Apr. 8, 2021 (CN) 202110387270.2

(51) Int. Cl. A61K 31/445; A61K 9/00; A61K 9/14; A61K 47/02; A61K 47/10; A61K 47/38

(52) U.S. Cl. CPC A61K 31/445; A61K 9/0019; A61K 9/14; A61K 47/02; A61K 47/10; A61K 47/38

(57) ABSTRACT

The present disclosure relates to a ropivacaine suspension injection, and a preparation method therefor. Specifically, the ropivacaine suspension injection of the present disclosure comprises ropivacaine and a pharmaceutically acceptable excipient, wherein the median particle size D50 of the suspension injection is within the range of 1 μm to 40 μm. The ropivacaine suspension injection of the present disclosure can continuously release a drug in vivo, and has an analgesic effect of no less than 12 h, preferably, an analgesic effect of no less than 24 h, and more preferably, an analgesic effect of no less than 72 h.

### TECHNICAL FIELD

The present disclosure relates to a ropivacaine suspension injection, and a preparation method therefor.

### BACKGROUND ART

Postoperative pain, which is an acute pain that occurs immediately after a surgery and usually lasts no more than 7 days, is substantially an acute nociceptive pain. It is also the most common acute pain that most urgently needs to be addressed. If not fully controlled in the initial state, postoperative pain can easily develop into chronic postoperative pain. Opioids are commonly used in clinical practice to treat postoperative pain, but they have adverse reactions such as respiratory depression and addiction. Local anesthetics (including procaine, lidocaine, tetracaine, bupivacaine, and ropivacaine) are also the most important clinical analgesics.

However, the existing local anesthetics have a relatively short effective duration of action, usually lasting less than 7 hours. Therefore, a device for continuous incisional analgesia is used clinically to instill amide local anesthetics in the wound to maintain a given therapeutic concentration. However, this device still has certain shortcomings. For example, a medicine storage bag must be carried around, which can be inconvenient for the patient: the placement of an osmotic catheter in the body can lead to increased local irritation, resulting in certain complications; and removing the osmotic catheter after treatment can be challenging. Therefore, the development of long-acting local anesthetics has currently become a hot research topic.

As one of the safest peripheral nerve block anesthetics in the market, ropivacaine is widely used in the management of acute pain. With regard to the mechanism of action, ropivacaine plays an anesthetic role by blocking nerve excitation and conduction through inhibiting sodium ion channels in neurons. Currently, ropivacaine hydrochloride injection is widely used in the market, and its analgesic effect lasts 3-6 hours after a single intrathecal injection: however, postoperative pain requires management for several days. Clinically, non-continuous multiple injections of local anesthetics, continuous drug infusion through epidural space or peripheral nerve catheterization and other methods can be used to achieve postoperative analgesia that lasts several days. Continuous infusion of local anesthetics can effectively control pain, but it requires relatively expensive equipment and continuous monitoring, and there is a risk of catheter displacement and complications (such as local tissue necrosis, cellulitis, and surgical wound infection) due to long-term catheter indwelling. In addition, frequent dosing can also result in increased plasma drug concentration fluctuations and decreased patient compliance, cause pain and inconvenience to patients, and lead to serious side effects.

In the field of scientific research, attempts have been made to use sustained-release gel preparations containing ropivacaine, but the sustained-release gel will swell after subcutaneous injection, causing tissue stretching and affecting both drug absorption and patient experience. Therefore, it is desirable in the art to provide a sustained-release ropivacaine preparation that not only achieves analgesic effects but also provides better patient experience or compliance.

### SUMMARY OF THE INVENTION

One embodiment of the present disclosure may provide a ropivacaine suspension injection comprising ropivacaine and a pharmaceutically acceptable excipient, wherein the median particle size D50 of the suspension injection is within the range of 1 μm to 40 μm.

One embodiment of the present disclosure may provide a method for preparing the ropivacaine suspension injection of the present disclosure, which method comprises the following steps:
(1) mixing ropivacaine and the pharmaceutically acceptable excipient to obtain mixture 1;
(2) subjecting the mixture 1 to shearing at a rotational speed of 3000-10000 rpm for 5 minutes or longer to obtain mixture 2;
(3) homogenizing the mixture 2 under a pressure of 20-2000 bar for one or more times to obtain mixture 3;
(4) optionally, adjusting the pH of the mixture 3;
(5) optionally, diluting the resulting mixture to a constant volume.

One embodiment of the present disclosure may provide a solid composition obtained by drying the ropivacaine suspension injection according to the present disclosure.

One embodiment of the present disclosure may provide the use of the ropivacaine suspension injection according to the present disclosure or the solid composition according to the present disclosure in the manufacture of a medicament for treating or preventing pain.

### DETAILED DESCRIPTION OF EMBODIMENTS

Unless otherwise specified, all numbers representing content, concentration, ratio, weight, volume, rotational speed, pressure, particle size, percentage, technical effect, etc. as used in the present description and claims should be understood in any case as being modified by the term “about” or “approximately”. Accordingly, unless indicated to the contrary, numerical parameters as set forth in the following description and attached claims are approximations. Unless otherwise specified, the terms used herein have the meanings commonly understood by those skilled in the pertinent technical field. For those skilled in the art, each numerical parameter may vary depending upon the desired properties and effects sought to be obtained by the present disclosure and should be construed in light of the significant figures of digits and ordinary rounding techniques or in a manner understood by those skilled in the art.

Although the broad range of the numerical values and the parameters which are approximations of the present disclosure are as set forth herein, the numerical values as set forth in the specific examples are given as precisely as possible. However, any numerical value inherently contains certain errors, which are inevitably caused by the standard deviation found in their respective test measurements. Every numerical range given throughout the present description will include every narrower numerical range that falls within such a broader numerical range, as if such narrower numerical ranges are all expressly written herein.

When used herein, the expression “A and/or B” includes three cases: (1) A; (2) B; and (3) A and B. The expression “A, B and/or C” includes seven cases: (1) A; (2) B; (3) C; (4) A and B; (5) A and C; (6) B and C; and (7) A, B and C. The meaning of similar expressions can be deduced in the same way.

As used herein, the term “median particle size D50” or “D50” refers to the particle size at which the cumulative distribution of particles is 50%, that is, the volume content of particles smaller than this particle size accounts for 50% of the total particle volume.

As used herein, the term “D10” refers to the particle size at which the cumulative distribution of particles is 10%, that is, the volume content of particles smaller than this particle size accounts for 10% of the total particle volume.

As used herein, the term “D90” refers to the particle size at which the cumulative distribution of particles is 90%, that is, the volume content of particles smaller than this particle size accounts for 90% of the total particle volume.

Due to low solubility in water, ropivacaine can be prepared as a suspension injection. For a ropivacaine suspension injection, if the particle size is too small, it will lead to a rapid release such that the effect of sustained release over an extended period cannot be achieved; if the particle size is too large, ropivacaine will be unevenly dispersed and accumulated in large quantities at the dosing site, resulting in multiple releases and unstable plasma drug concentration. In addition, a too large particle size will also affect the syringeability of a preparation, making it inconvenient for clinical dosing.

The present disclosure provides a ropivacaine suspension injection which is stably released in vivo over an extended period. The suspension injection can not only prolong the analgesic effect for postoperative pain but also provide convenience for physicians and patients, thereby achieving good medication compliance. The ropivacaine suspension injection of the present disclosure can continuously release a drug in vivo, and has an analgesic effect of no less than 12 h, preferably, an analgesic effect of no less than 24 h, and more preferably, an analgesic effect of no less than 72 h. In addition, compared with the currently available ropivacaine preparations in the market, the ropivacaine suspension injection of the present disclosure has a greatly reduced cost (the cost is only about ⅓ to ¼ of the cost of the commercially available ropivacaine preparations), which can benefit more patients suffering from pain and reduce their burden of drug expenses.

One embodiment of the present disclosure may provide a ropivacaine suspension injection comprising ropivacaine and a pharmaceutically acceptable excipient, wherein the median particle size D50 of the suspension injection is within the range of about 1 μm to about 40 μm.

In some embodiments of the present disclosure, the suspension injection can have a particle size distribution within the ranges of: D10: about 1 μm to about 10 μm, D50: about 1 μm to about 40 μm, and D90: about 5 μm to about 100 μm.

One embodiment of the present disclosure may provide a ropivacaine suspension injection comprising ropivacaine and a pharmaceutically acceptable excipient, wherein the median particle size D50 of the suspension injection is within the range of about 3 μm to about 40 μm.

In some embodiments of the present disclosure, the suspension injection can have a particle size distribution within the ranges of: D10: about 1 μm to about 10 μm, D50: about 3 μm to about 40 μm, and D90: about 5 μm to about 100 μm.

One embodiment of the present disclosure may provide a ropivacaine suspension injection comprising ropivacaine and a pharmaceutically acceptable excipient, wherein the median particle size D50 of the suspension injection is within the range of about 5 μm to about 20 μm.

In some embodiments of the present disclosure, the suspension injection can have a particle size distribution within the ranges of: D10: about 2 μm to about 8 μm, D50: about 5 μm to about 20 μm, and D90: about 10 μm to about 60 μm.

In some embodiments of the present disclosure, the median particle size D50 of the suspension injection can be within the range of about 9 μm to about 40 μm.

In some embodiments of the present disclosure, the suspension injection can have a particle size distribution within the ranges of: D10: about 2.5 μm to about 10 μm, D50: about 9 μm to about 40 μm, and D90: about 20 μm to about 100 μm.

In some embodiments of the present disclosure, the median particle size D50 of the suspension injection can be within the range of about 12 μm to about 37 μm.

In some embodiments of the present disclosure, the suspension injection can have a particle size distribution within the ranges of: D10: about 4 μm to about 10 μm, D50: about 12 μm to about 37 μm, and D90: about 30 μm to about 90 μm.

In some embodiments of the present disclosure, the pharmaceutically acceptable excipient can comprise a suspending agent, an isoosmotic adjusting agent and a surfactant. In some embodiments of the present disclosure, the pharmaceutically acceptable excipient can further comprise a buffering agent. In some embodiments of the present disclosure, the pharmaceutically acceptable excipient can further comprise a pH regulator. In some embodiments of the present disclosure, the pharmaceutically acceptable excipient can further comprise a preservative.

In some embodiments of the present disclosure, the suspending agent can be selected from one or more of carboxymethyl cellulose and a sodium salt thereof, hydroxypropyl cellulose and a sodium salt thereof, hydroxypropyl methylcellulose and a sodium salt thereof, methyl cellulose and a sodium salt thereof, hydroxyethyl cellulose and a sodium salt thereof, sodium hyaluronate and polyvinylpyrrolidone. In some embodiments of the present disclosure, the suspending agent can be sodium carboxymethyl cellulose.

In some embodiments of the present disclosure, the isoosmotic adjusting agent can be selected from one or more of sodium chloride, mannitol, sorbitol, glucose, sucrose, fructose, and lactose. In some embodiments of the present disclosure, the isoosmotic adjusting agent can be sodium chloride and/or mannitol. In some embodiments of the present disclosure, the isoosmotic adjusting agent can be sodium chloride. In some embodiments of the present disclosure, the isoosmotic adjusting agent can be mannitol.

In some embodiments of the present disclosure, the surfactant can be selected from one or more of poloxamer, lecithin, polysorbate 20, polysorbate 40, polysorbate 60, polysorbate 65, polysorbate 80, polysorbate 85, polyoxyethylenated castor oil, polyoxyethylenated hydrogenated castor oil, polyvinylpyrrolidone, polyethylene glycols and polyethylene glycol 15-hydroxystearate. In some embodiments of the present disclosure, the surfactant can be poloxamer and/or lecithin. In some embodiments of the present disclosure, the surfactant can be poloxamer 188. In some embodiments of the present disclosure, the surfactant can be egg yolk lecithin.

In some embodiments of the present disclosure, the buffering agent can be selected from one or more of a phosphate buffer system, an acetate buffer system, a citrate buffer system, and a tris(hydroxymethyl)aminomethane buffer system. In some embodiments of the present disclosure, the buffering agent can be NaH₂PO₄/Na₂HPO₄.

In some embodiments of the present disclosure, the pH regulator can be selected from one or more of fumaric acid, metatartaric acid, citric acid, lactic acid, malic acid, tartaric acid, acetic acid, adipic acid, phosphoric acid, hydrochloric acid, calcium hydroxide, potassium hydroxide, sodium hydroxide, monosodium fumarate, sodium citrate, potassium citrate, monosodium citrate, phosphate, calcium sulfate, calcium lactate, and sodium acetate. In some embodiments of the present disclosure, the buffering agent can be used as a pH regulator.

In some embodiments of the present disclosure, the preservative can be selected from one or more of benzoic acid, benzalkonium chloride, benzyl alcohol, butylated hydroxyanisole, butylated hydroxytoluene, chlorobutanol, a gallate, methyl hydroxybenzoate, ethylene diamine tetraacetic acid and a salt thereof, chlorocresol, m-cresol, benzethonium chloride, myristyl-γ-methylpyridine chloride, phenylmercury acetate, and thimerosal.

In some embodiments of the present disclosure, every 100 mL of the suspension injection can comprise about 0.1-20 g ropivacaine: about 1-10 g ropivacaine: about 1.5-5 g ropivacaine: about 1.8-4 g ropivacaine: or about 2-3.5 g ropivacaine:

in some embodiments of the present disclosure, every 100 mL of the suspension injection can comprise about 0.1-2 g suspending agent: about 0.2-1 g suspending agent: about 0.3-0.8 g suspending agent: about 0.4-0.75 g suspending agent: about 0.5-0.63 g suspending agent: or about 0.5-0.63 g sodium carboxymethyl cellulose.

In some embodiments of the present disclosure, every 100 mL of the suspension injection can comprise about 0.1-10 g isoosmotic adjusting agent: about 0.3-7.5 g isoosmotic adjusting agent: about 0.5-7.5 g isoosmotic adjusting agent: about 0.5-0.8 g sodium chloride: about 0.6-0.7 g sodium chloride: about 4-6 g mannitol: or about 4.5-5.5 g mannitol:

in some embodiments of the present disclosure, every 100 mL of the suspension injection can comprise about 0.1-10 g surfactant: about 0.25-5 g surfactant: about 0.35-4 g surfactant: about 0.35-0.65 g poloxamer; about 0.4-0.6 g poloxamer; about 0.4-0.6 g poloxamer 188; about 1.5-4 g lecithin; about 2-3 g lecithin; or about 2-3 g egg yolk lecithin.

In some embodiments of the present disclosure, every 100 mL of the suspension injection can comprise:
about 0.1-20 g ropivacaine:
about 0.1-2 g suspending agent;
about 0.1-10 g isoosmotic adjusting agent; and
about 0.1-10 g surfactant.

In some embodiments of the present disclosure, every 100 mL of the suspension injection can comprise:
about 1-10 g ropivacaine:
about 0.2-1 g suspending agent:
about 0.3-7.5 g isoosmotic adjusting agent; and
about 0.25-5 g surfactant.

In some embodiments of the present disclosure, every 100 mL of the suspension injection can comprise:
about 1.5-5 g ropivacaine:
about 0.3-0.8 g suspending agent;
about 0.5-7.5 g isoosmotic adjusting agent; and
about 0.35-4 g surfactant.

In some embodiments of the present disclosure, every 100 mL of the suspension injection can comprise:
about 1.8-4 g ropivacaine:
about 0.4-0.75 g suspending agent:
about 0.5-0.8 g sodium chloride or about 4-6 g mannitol; and
about 0.35-0.65 g poloxamer or about 1.5-4 g lecithin.

In some embodiments of the present disclosure, every 100 mL of the suspension injection can comprise:
about 2-3.5 g ropivacaine;
about 0.5-0.63 g suspending agent;
about 0.6-0.7 g sodium chloride or about 4.5-5.5 g mannitol; and
about 0.4-0.6 g poloxamer or about 2-3 g lecithin.

In some embodiments of the present disclosure, every 100 mL of the suspension injection can comprise:
about 2-3.5 g ropivacaine:
about 0.5-0.63 g sodium carboxymethyl cellulose:
about 0.6-0.7 g sodium chloride or about 4.5-5.5 g mannitol; and
about 0.4-0.6 g poloxamer 188 or about 2-3 g egg yolk lecithin.

In some embodiments of the present disclosure, every 100 mL of the suspension injection can further comprise a buffering agent NaH₂PO₄/Na₂HPO₄ and water for injection.

In some embodiments of the present disclosure, the pH of the suspension injection can be about 5-9, preferably about 6-8, preferably about 6.5-8, more preferably about 7-8, more preferably about 7.2-7.8, more preferably about 7.2-7.6, more preferably about 7.3-7.5, and more preferably about 7.4.

In some embodiments of the present disclosure, the suspension injection is administered by subcutaneous, intradermal or intramuscular injection.

The above-mentioned various embodiments and preferences for the ropivacaine suspension injection of the present disclosure can be combined with each other (as long as they are not inherently contradictory to each other), and the various embodiments formed by the combination are considered as a part of the present disclosure.

One embodiment of the present disclosure can provide a method for preparing the ropivacaine suspension injection of the present disclosure, which method comprises the following steps:
(1) mixing ropivacaine and the pharmaceutically acceptable excipient to obtain mixture 1;
(2) subjecting the mixture 1 to shearing at a rotational speed of 3000-10000 rpm for 5 minutes or longer to obtain mixture 2;
(3) homogenizing the mixture 2 under a pressure of 20-2000 bar for one or more times to obtain mixture 3;
(4) optionally, adjusting the pH of the mixture 3;
(5) optionally, diluting the resulting mixture to a constant volume.

In some embodiments of the present disclosure, step (1) comprises:
(1.1) dissolving the suspending agent, the isoosmotic adjusting agent and the surfactant in water for injection to obtain solution 1;
(1.2) adding ropivacaine to the solution 1 to obtain mixture 1.

In some embodiments of the present disclosure, the mixture 1 is subjected to shearing at a rotational speed of 4000-9000 rpm for 5 minutes or longer. In some embodiments of the present disclosure, the mixture 1 is subjected to shearing at a rotational speed of 4500-8500 rpm for 5 minutes or longer. In some embodiments of the present disclosure, the mixture 1 is subjected to shearing at a rotational speed of 5000-8000 rpm for 5 minutes or longer. In some embodiments of the present disclosure, the mixture 1 is subjected to shearing at a rotational speed of 5000-8500 rpm for 5 minutes or longer. In some embodiments of the present disclosure, the mixture 1 is subjected to shearing at a rotational speed of 5000-9000 rpm for 5 minutes or longer. In some embodiments of the present disclosure, the mixture 1 is subjected to shearing at a rotational speed of 6000-9000 rpm for 5 minutes or longer. In some embodiments of the present disclosure, the mixture 1 is subjected to shearing at a rotational speed of 6000-10000 rpm for 5 minutes or longer. In some embodiments of the present disclosure, the mixture 1 is subjected to shearing at a rotational speed of 7000-9000 rpm for 5 minutes or longer. In some embodiments of the present disclosure, the mixture 1 is subjected to shearing at a rotational speed of 8000-9000 rpm for 5 minutes or longer. In some embodiments of the present disclosure, the mixture 1 is subjected to shearing at a rotational speed of 7000-8000 rpm for 5 minutes or longer. In some embodiments of the present disclosure, the mixture 1 is subjected to shearing at a rotational speed of 7500-8500 rpm for 5 minutes or longer.

In some embodiments of the present disclosure, the shearing lasts 6 minutes or longer. In some embodiments of the present disclosure, the shearing lasts 7 minutes or longer. In some embodiments of the present disclosure, the shearing lasts 8 minutes or longer. In some embodiments of the present disclosure, the shearing lasts 9 minutes or longer. In some embodiments of the present disclosure, the shearing lasts 10 minutes or longer. In some embodiments of the present disclosure, the shearing lasts 12 minutes or longer. In some embodiments of the present disclosure, the shearing lasts 15 minutes or longer. In some embodiments of the present disclosure, the shearing lasts 18 minutes or longer. In some embodiments of the present disclosure, the shearing lasts 20 minutes or longer. In some embodiments of the present disclosure, the shearing lasts 25 minutes or longer. In some embodiments of the present disclosure, the shearing lasts 30 minutes or longer. In some embodiments of the present disclosure, the shearing lasts 1 hour or longer. In some embodiments of the present disclosure, the shearing lasts 2 hours or longer. In some embodiments of the present disclosure, the mixture 1 is subjected to shearing at a rotational speed of 6000-10000 rpm for 10 minutes or longer. In some embodiments of the present disclosure, the mixture 1 is subjected to shearing at a rotational speed of 7000-9000 rpm for 10 minutes or longer. In some embodiments of the present disclosure, the mixture 1 is subjected to shearing at a rotational speed of 7500-8500 rpm for 10 minutes or longer.

In some embodiments of the present disclosure, the mixture 1 is subjected to shearing at a rotational speed of 6000-10000 rpm for 20 minutes or longer. In some embodiments of the present disclosure, the mixture 1 is subjected to shearing at a rotational speed of 7000-9000 rpm for 20 minutes or longer. In some embodiments of the present disclosure, the mixture 1 is subjected to shearing at a rotational speed of 7500-8500 rpm for 20 minutes or longer.

In some embodiments of the present disclosure, the mixture 1 is subjected to shearing at a rotational speed of 6000-10000 rpm for 30 minutes or longer. In some embodiments of the present disclosure, the mixture 1 is subjected to shearing at a rotational speed of 7000-9000 rpm for 30 minutes or longer. In some embodiments of the present disclosure, the mixture 1 is subjected to shearing at a rotational speed of 7500-8500 rpm for 30 minutes or longer.

In some embodiments of the present disclosure, the mixture 2 is homogenized under a pressure of 30-1500 bar for one or more times. In some embodiments of the present disclosure, the mixture 2 is homogenized under a pressure of 40-1000 bar for one or more times. In some embodiments of the present disclosure, the mixture 2 is homogenized under a pressure of 40-500 bar for one or more times. In some embodiments of the present disclosure, the mixture 2 is homogenized under a pressure of 40-400 bar for one or more times. In some embodiments of the present disclosure, the mixture 2 is homogenized under a pressure of 40-300 bar for one or more times. In some embodiments of the present disclosure, the mixture 2 is homogenized under a pressure of 40-200 bar for one or more times. In some embodiments of the present disclosure, the mixture 2 is homogenized under a pressure of 40-150 bar for one or more times. In some embodiments of the present disclosure, the mixture 2 is homogenized under a pressure of 40-120 bar for one or more times. In some embodiments of the present disclosure, the mixture 2 is homogenized under a pressure of 40-110 bar for one or more times. In some embodiments of the present disclosure, the mixture 2 is homogenized under a pressure of 40-100 bar for one or more times. In some embodiments of the present disclosure, the mixture 2 is homogenized under a pressure of 45-500 bar for one or more times. In some embodiments of the present disclosure, the mixture 2 is homogenized under a pressure of 45-400 bar for one or more times. In some embodiments of the present disclosure, the mixture 2 is homogenized under a pressure of 45-300 bar for one or more times. In some embodiments of the present disclosure, the mixture 2 is homogenized under a pressure of 45-200 bar for one or more times. In some embodiments of the present disclosure, the mixture 2 is homogenized under a pressure of 45-150 bar for one or more times. In some embodiments of the present disclosure, the mixture 2 is homogenized under a pressure of 45-120 bar for one or more times. In some embodiments of the present disclosure, the mixture 2 is homogenized under a pressure of 45-110 bar for one or more times. In some embodiments of the present disclosure, the mixture 2 is homogenized under a pressure of 45-100 bar for one or more times. In some embodiments of the present disclosure, the mixture 2 is homogenized under a pressure of 50-500 bar for one or more times. In some embodiments of the present disclosure, the mixture 2 is homogenized under a pressure of 50-400 bar for one or more times. In some embodiments of the present disclosure, the mixture 2 is homogenized under a pressure of 50-300 bar for one or more times. In some embodiments of the present disclosure, the mixture 2 is homogenized under a pressure of 50-200 bar for one or more times. In some embodiments of the present disclosure, the mixture 2 is homogenized under a pressure of 50-150 bar for one or more times. In some embodiments of the present disclosure, the mixture 2 is homogenized under a pressure of 50-120 bar for one or more times. In some embodiments of the present disclosure, the mixture 2 is homogenized under a pressure of 50-110 bar for one or more times. In some embodiments of the present disclosure, the mixture 2 is homogenized under a pressure of 50-100 bar for one or more times.

In some embodiments of the present disclosure, the homogenization is performed 1-10 times. In some embodiments of the present disclosure, the homogenization is performed 1-8 times. In some embodiments of the present disclosure, the homogenization is performed 1-7 times. In some embodiments of the present disclosure, the homogenization is performed 1-6 times. In some embodiments of the present disclosure, the homogenization is performed 1-5 times. In some embodiments of the present disclosure, the homogenization is performed 1-4 times. In some embodiments of the present disclosure, the homogenization is performed 1-3 times. In some embodiments of the present disclosure, the homogenization is performed 1-2 times. In some embodiments of the present disclosure, the homogenization is performed once.

One embodiment of the present disclosure may provide a solid composition obtained by drying the suspension injection according to the present disclosure. Drying can be performed using methods commonly known in the art, including but not limited to freeze drying, spray drying, spray freeze drying, or other conventional drying means.

One embodiment of the present disclosure may provide the use of the suspension injection according to the present disclosure or the solid composition according to the present disclosure in the manufacture of a medicament for treating or preventing pain.

The above-mentioned various embodiments and preferences for the ropivacaine suspension injection of the present disclosure are also applicable to the preparation method, the solid composition, and the use of the present disclosure, and these embodiments and preferences can also be combined with each other (as long as they are not inherently contradictory to each other), and the various embodiments formed by the combination are considered as a part of the present application.

The technical solution of the present disclosure will be more clearly and explicitly described by way of illustration in combination with examples. It should be understood that these examples are only for illustrative purposes and not intended to limit the protection scope of the present disclosure. The protection scope of the present disclosure is only defined by the claims.

### EXAMPLES

Among the materials used in the examples, ropivacaine (CAS number: 84057-95-4) was purchased from Xianju Pharmaceutical Co., Ltd.; sodium carboxymethyl cellulose was purchased from Ashland Company; poloxamer 188 was purchased from Merck KGaA; egg yolk lecithin was purchased from Kewpie Group (Japan); mannitol was purchased from Roquette Company; sodium chloride was purchased from Jiangsu Province Qinfen Pharmaceutical Co., Ltd.; and sodium dihydrogen phosphate and disodium hydrogen phosphate were purchased from Nanjing Chemical Reagent Co., Ltd.

### Example 1: Preparation of Ropivacaine Suspension Injection I

Sodium carboxymethyl cellulose (0.63 g), sodium chloride (0.66 g) and egg yolk lecithin (2.67 g) were weighed and dissolved in water for injection (90 ml). Upon complete dissolution, ropivacaine (2.0 g) was weighed and added to the above-mentioned solution, and a high shear was performed at a rotational speed of 8000 rpm for 10 minutes to fully disperse ropivacaine. Subsequently, the mixture was homogenized with a high-pressure homogenizer (Model: GEA Panda Plus) under a pressure of 1900 bar for 3 times to obtain a suspension. The particle size of the suspension was measured by a Malvern 3000 particle size analyzer, and the particle size results were as follows: D10: 1.65 μm, D50: 3.80 μm, and D90: 7.80 μm. An appropriate amount of sodium dihydrogen phosphate and disodium hydrogen phosphate was weighed to adjust the suspension to pH 7.4, and the resulting suspension was diluted to a constant volume of 100 ml with water for injection.

### Example 2: Preparation of Ropivacaine Suspension Injection II

Mannitol (5 g), sodium carboxymethyl cellulose (0.5 g) and poloxamer 188 (0.5 g) were weighed and dissolved in water for injection (90 ml). Upon complete dissolution, ropivacaine (3.0 g) was weighed and added to the above-mentioned solution, and a high shear was performed at a rotational speed of 8000 rpm for 10 minutes to fully disperse ropivacaine. Subsequently, the mixture was homogenized with a high-pressure homogenizer (Model: ATS AH-2010) under a pressure of 400 bar for 4 times to obtain a suspension. The particle size of the suspension was measured by a Malvern 3000 particle size analyzer, and the particle size results were as follows: D10: 2.79 μm, D50: 9.07 μm, and D90: 20.6 μm. An appropriate amount of sodium dihydrogen phosphate and disodium hydrogen phosphate was weighed to adjust the suspension to pH 7.4, and the resulting suspension was diluted to a constant volume of 100 ml with water for injection.

### Example 3: Preparation of Ropivacaine Suspension Injection III

Sodium carboxymethyl cellulose (0.945 g), sodium chloride (0.99 g) and poloxamer 188 (0.75 g) were weighed and dissolved in water for injection (140 ml). Upon complete dissolution, ropivacaine (4.5 g) was weighed and added to the above-mentioned solution, and a high shear was performed at a rotational speed of 8000 rpm for 10 minutes to fully disperse ropivacaine.

Subsequently, the mixture was homogenized once with a high-pressure homogenizer (Model: GEA Panda Plus) under a pressure of 150 bar to obtain a suspension. The particle size of the suspension was measured by a Malvern 3000 particle size analyzer, and the particle size results were as follows: D10: 4.81 μm, D50: 14.5 μm, D90: 32.1 μm. An appropriate amount of sodium dihydrogen phosphate and disodium hydrogen phosphate was weighed to adjust the suspension to pH 7.4, and the resulting suspension was diluted to a constant volume of 150 ml with water for injection.

### Example 4: Preparation of Ropivacaine Suspension Injection IV

Sodium carboxymethyl cellulose (0.945 g), sodium chloride (0.99 g) and poloxamer 188 (0.75 g) were weighed and dissolved in water for injection (140 ml). Upon complete dissolution, ropivacaine (4.5 g) was weighed and added to the above-mentioned solution, and a high shear was performed at a rotational speed of 8000 rpm for 10 minutes to fully disperse ropivacaine. Subsequently, the mixture was homogenized once with a high-pressure homogenizer (Model: GEA Panda Plus) under a pressure of 50 bar to obtain a suspension. The particle size of the suspension was measured by a Malvern 3000 particle size analyzer, and the particle size results were as follows: D10: 9.3 μm, D50: 34.5 μm, and D90: 85.5 μm. An appropriate amount of sodium dihydrogen phosphate and disodium hydrogen phosphate was weighed to adjust the suspension to pH 7.4, and the resulting suspension was diluted to a constant volume of 150 ml with water for injection.

### Example 5: Pharmacokinetics (PK) Study of Ropivacaine Suspension Injections I-IV in SD Rats

15 healthy male SD rats (Shanghai SLAC Laboratory Animal Co., Ltd.) were selected and divided into the following 5 groups (3 rats/group): experimental group I with ropivacaine suspension injection I; experimental group II with ropivacaine suspension injection II; experimental group III with ropivacaine suspension injection III: experimental group IV with ropivacaine suspension injection IV; and control group I with a marketed ropivacaine hydrochloride injection (Zhejiang Xianju Pharmaceutical Co., Ltd.). The detailed dosing regimen is shown below.

| Group | Dosage for SD rats | | Dosing site |
|---|---|---|---|
| Experimental group I | 37.5 | mg/kg | Muscle |
| Experimental group II | 37.5 | mg/kg | Muscle |
| Experimental group III | 37.5 | mg/kg | Muscle |
| Experimental group IV | 37.5 | mg/kg | Muscle |
| Control group I | 18.75 | mg/kg | Muscle |

Venous blood was collected at specific time points post-dosing, and a summary of the plasma drug concentration and pharmacokinetic parameters (average values) of ropivacaine is shown below.

| | Group | | | | |
|---|---|---|---|---|---|
| | Experimental | Experimental | Experimental | Experimental | Control |
| | group I | group II | group III | group IV | group I |
| Time | Plasma drug concentration (ng/ml) | | | | |
| Prior to | 0 | 0 | 0 | 0 | 0 |
| dosing | | | | | |
| 0.25 h | 277.91 | 378.31 | 170.11 | 146.20 | 1641.70 |
| 0.5 h | / | 545.60 | / | / | 1900.44 |
| 1 h | / | 539.42 | / | / | 1707.97 |
| 1.5 h | / | 697.67 | / | / | 1389.69 |
| 2 h | 456.03 | 691.95 | 605.31 | 394.67 | 1021.37 |
| 2.5 h | / | 674.29 | / | / | 716.79 |
| 4 h | 314.72 | 522.64 | 405.88 | 326.31 | 182.72 |
| 6 h | 212.87 | 510.74 | 383.68 | 344.80 | 19.53 |
| 10 h | 86.49 | 237.14 | 234.38 | 246.55 | ND |
| 16 h | 25.23 | / | 116.60 | 122.62 | / |
| 24 h | ND | 75.49 | 68.22 | 95.28 | ND |
| 32 h | ND | 50.91 | 53.50 | 64.97 | ND |
| 48 h | ND | ND | ND | 20.74 | ND |
| 56 h | / | ND | ND | ND | ND |
| 72 h | / | ND | ND | ND | ND |
| 80 h | / | ND | ND | ND | ND |
| 96 h | / | ND | ND | ND | ND |

Average pharmacokinetic parameters

| | | | | | |
|---|---|---|---|---|---|
| Tₘₐₓ (h) | 2.00 | 2.17 | 2.00 | 2.67 | 0.50 |
| Cₘₐₓ | 456.03 | 768.97 | 605.31 | 479.58 | 1900.44 |
| (ng/ml) | | | | | |
| AUC₀₋ₜ | 2909.17 | 7553.08 | 6521.84 | 6372.02 | 4238.68 |
| (ng/(ml · h)) | | | | | |
| AUC_{0-∞} | 3031.00 | 8347.70 | 6645.35 | 6668.81 | 4259.02 |
| (ng/(ml · h)) | | | | | |
| MRT₀₋ₜ | 5.3344 | 12.0413 | 12.3003 | 15.8409 | 1.5682 |
| (h) | | | | | |
| T_{1/2} | 3.2869 | 8.45 | 8.50 | 9.86 | 0.6347 |
| (h) | | | | | |

Note:
“/” indicates that no sample was collected at this time point; and “ND” indicates not detected.

The results show that after the male rats were dosed via intramuscular injection, the plasma drug concentration in control group I, which received the true solution, was undetectable at 10 hours post-dosing. In contrast, in example I, the plasma drug concentration remained detectable at 16 hours post-dosing: in examples II and III, the plasma drug concentration remained detectable at 32 hours post-dosing; and in example IV, the plasma drug concentration remained detectable at 48 hours post-dosing. Compared with control group I, experimental groups I-IV showed significant sustained-release effects in terms of the average pharmacokinetic parameters, and also exhibited significantly prolonged MRT₀₋ₜ, T_{1/2}, and Tₘₐₓ. In addition, the Cₘₐₓ of experimental groups I-IV was significantly lower than that of control group I, indicating a reduction in toxic side effects. It can be seen therefrom that the ropivacaine suspension injection of the present disclosure has a good long-acting analgesic effect.

### Example 6: Preparation of Ropivacaine Suspension Injection V

Sodium carboxymethyl cellulose (0.945 g), sodium chloride (0.99 g) and poloxamer 188 (0.75 g) were weighed and dissolved in water for injection (140 ml). Upon complete dissolution, ropivacaine (5.25 g) was weighed and added to the above-mentioned solution, and a high shear was performed at a rotational speed of 8000 rpm for 10 minutes to fully disperse ropivacaine. Subsequently, the mixture was homogenized once with a high-pressure homogenizer (Model: GEA Panda Plus) under a pressure of 100 bar to obtain a suspension. The particle size of the suspension was measured by a Malvern 3000 particle size analyzer, and the particle size results were as follows: D10: 5.99 μm, D50: 19.2 μm, and D90: 45.0 μm. An appropriate amount of sodium dihydrogen phosphate and disodium hydrogen phosphate was weighed to adjust the suspension to pH 7.4, and the resulting suspension was diluted to a constant volume of 150 ml with water for injection.

### Example 7: Preparation of Ropivacaine Suspension Injection VI

Sodium carboxymethyl cellulose (0.945 g), sodium chloride (0.99 g) and poloxamer 188 (0.75 g) were weighed and dissolved in water for injection (140 ml). Upon complete dissolution, ropivacaine (5.25 g) was weighed and added to the above-mentioned solution, and a high shear was performed at a rotational speed of 8000 rpm for 10 minutes to fully disperse ropivacaine. Subsequently, the mixture was homogenized once with a high-pressure homogenizer (Model: GEA Panda Plus) under a pressure of 50 bar to obtain a suspension. The particle size of the suspension was measured by a Malvern 3000 particle size analyzer, and the particle size results were as follows: D10: 8.66 μm, D50: 30.9 μm, and D90: 77.8 μm. An appropriate amount of sodium dihydrogen phosphate and disodium hydrogen phosphate was weighed to adjust the suspension to pH 7.4, and the resulting suspension was diluted to a constant volume of 150 ml with water for injection.

### Example 8: Pharmacokinetics (PK) Study of Ropivacaine Suspension Injections V-VI in Bama Mini-Pigs

9 healthy male Bama mini-pigs (Wujiang Tianyu Biotechnology Co., Ltd.) were selected and divided into the following 3 groups (3 mini-pigs/group): experimental group V with ropivacaine suspension injection V; experimental group VI with ropivacaine suspension injection VI; and control group II with a marketed ropivacaine hydrochloride injection (Zhejiang Xianju Pharmaceutical Co., Ltd.). The detailed dosing regimen is shown below.

| Group | Dosage for Bama mini-pigs | Dosing site |
|---|---|---|
| Experimental group V | 8.0 mg/kg | Subcutaneous site |
| Experimental group VI | 8.0 mg/kg | Subcutaneous site |
| Control group II | 1.7 mg/kg | Subcutaneous site |

Venous blood was collected at specific time points post-dosing, and a summary of the plasma drug concentration and pharmacokinetic parameters (average values) of ropivacaine is shown below.

| | Group | | |
|---|---|---|---|
| | Experimental group V | Experimental group VI | Control group II |
| Time | Plasma drug concentration (ng/ml) | | |
| Prior to dosing | 0 | 0 | 0 |
| 0.25 h | 537.73 | 600.95 | 1717.32 |
| 1 h | 377.68 | 594.49 | 581.79 |
| 2 h | 318.36 | 339.33 | 258.83 |
| 4 h | 315.88 | 256.10 | 137.36 |
| 6 h | 552.98 | 339.11 | 111.57 |
| 10 h | 395.44 | 768.93 | 53.76 |
| 14 h | 431.69 | 274.31 | 20.65 |
| 24 h | 303.73 | 412.53 | 20.63 |
| 32 h | 259.85 | 362.58 | 2.46 |
| 48 h | 246.45 | 200.54 | ND |
| 56 h | 184.82 | 152.87 | ND |
| 72 h | 124.31 | 116.55 | ND |

Average pharmacokinetic parameters

| | | | |
|---|---|---|---|
| Tₘₐₓ (h) | 2.17 | 7.00 | 0.25 |
| Cₘₐₓ | 707.92 | 983.54 | 1717.32 |
| (ng/ml) | | | |
| AUC₀₋ₜ | 19992.96 | 21092.14 | 2728.48 |
| (ng/(ml · h)) | | | |
| AUC_{0-∞} | 26184.16 | 25339.31 | 2790.04 |
| (ng/(ml · h)) | | | |
| MRT₀₋ₜ | 51.0873 | 40.4274 | 3.9346 |
| (h) | | | |
| T_{1/2} | 31.8192 | 24.0815 | 3.7871 |
| (h) | | | |

Note:
“/” indicates that no sample was collected at this time point; and “ND” indicates not detected.

The results show that after the healthy male Bama mini-pigs were dosed via subcutaneous injection, the plasma drug concentration in control group II was undetectable at 48 hours post-dosing. However, in experimental groups V and VI, a high plasma drug concentration was still detected at 72 hours post-dosing. Compared with control group II, experimental groups V-VI showed significant sustained-release effects in terms of the average pharmacokinetic parameters, and also exhibited significantly prolonged MRT₀₋ₜ, T_{1/2}, and Tₘₐₓ. In addition, the Cₘₐₓ of experimental groups V-VI was significantly lower than that of control group II, indicating a reduction in toxic side effects. It can be seen therefrom that the ropivacaine suspension injection of the present disclosure has a good long-acting analgesic effect.

1. A ropivacaine suspension injection, comprising ropivacaine and a pharmaceutically acceptable excipient, wherein the median particle size D50 of the suspension injection is within the range of 1 μm to 40 μm.

2. The suspension injection of claim 1, wherein the median particle size D50 of the suspension injection is within the range of 3 μm to 40 μm.

3. The suspension injection of claim 2, wherein the median particle size D50 of the suspension injection is within the range of 5 μm to 20 μm.

4. The suspension injection of claim 1, wherein the pharmaceutically acceptable excipient comprises a suspending agent, an isoosmotic adjusting agent and a surfactant.

5. The suspension injection of claim 1, wherein every 100 mL of the suspension injection comprises:
0.1-20 g of ropivacaine;
0.1-2 g of the suspending agent;
0.1-10 g of the isoosmotic adjusting agent; and
0.1-10 g of the surfactant.

6. The suspension injection of claim 1, wherein the pH of the suspension injection is 5-9.

7. A method for preparing the suspension injection of claim 1, the method comprising the following steps:
(1) mixing ropivacaine and the pharmaceutically acceptable excipient to obtain mixture 1;
(2) subjecting the mixture 1 to shearing at a rotational speed of 3000-10000 rpm for 5 minutes or longer to obtain mixture 2; and
(3) homogenizing the mixture 2 under a pressure of 20-2000 bar for one or more times to obtain mixture 3.

8. The method of claim 7, wherein the step (1) comprises:
(1.1) dissolving the suspending agent, the isoosmotic adjusting agent and the surfactant in water for injection to obtain solution 1; and
(1.2) adding ropivacaine to the solution 1 to obtain the mixture 1.

9. A solid composition obtained by drying the suspension injection of claim 1.

10. (canceled)

11. The method of claim 7, wherein the method comprises step:
(4) adjusting the pH of the mixture 3.

12. The method of claim 11, wherein the method comprises step:
(5) diluting the resulting mixture to a constant volume.

13. A method of treating or preventing pain in a subject, the method comprises administering the suspension injection of claim 1 to a subject in need thereof.

14. A method of treating or preventing pain in a subject, the method comprises administering the solid composition of claim 9 to a subject in need thereof.
